(19) United States
(12) Patent Application Publication
(10) Pub. No.: US 2020/0207690 A1
(43) Pub. Date: Jul. 2, 2020

(54) METHOD FOR CONTROLLING THE CATALYTIC HYDROGENATION OF 1,4-BUTYNEDIOL VIA THE CONTENT OF CO AND/OR CH4 IN THE EXHAUST GAS STREAM

(71) Applicant: BASF SE, Ludwigshafen am Rhein (DE)

(72) Inventors: Rolf Pinkos, Ludwigshafen (DE); Jens Weiguny, Ludwigshafen (DE); Jens Baldamus, Ludwigshafen (DE); Michael Schwarz, Ludwigshafen (DE)

(21) Appl. No.: 16/644,731

(22) PCT Filed: Aug. 27, 2018
(86) PCT No.: PCT/EP2018/072999

§ 371 Date: Mar. 5, 2020

(30) Foreign Application Priority Data
Sep. 6, 2017 (EP) 17189578.2

(51) Int. Cl. C07C 29/17

(52) U.S. Cl. CPC C07C 29/172

(57) ABSTRACT

Described herein is a process for preparing butane-1,4-diol by catalytic hydrogenation of butyne-1,4-diol in a reaction zone with hydrogen in the presence of a heterogeneous hydrogenation catalyst, in which the content of at least one gas selected from CO and CH₄ in the offgas stream is measured and the content of the gas measured in the offgas stream is used for closed-loop control of the hydrogenation.

### CROSS-REFERENCE TO RELATED APPLICATIONS

This application is the U.S. National Stage filing of International Patent Application No. PCT/EP2018/072999, filed Aug. 27, 2018, which claims the benefit of priority to European Patent Application No. 17189578.2, filed Sep. 6, 2017, each of which are hereby incorporated by reference herein in their entirety.

### FIELD OF THE INVENTION

The present invention relates to a process for preparing butane-1,4-diol by catalytic hydrogenation of butyne-1,4-diol in a reaction zone with hydrogen in the presence of a heterogeneous hydrogenation catalyst, in which the content of at least one gas selected from CO and CH₄ in the offgas stream is measured and the content of the gas measured in the offgas stream is used for closed-loop control of the hydrogenation.

### BACKGROUND

In the chemical industry, catalytic hydrogenation is one of the most important reactions for the production of chemical products. The hydrogenation is preferably effected in the presence of heterogeneous catalysts which, by contrast with homogeneous catalysts, are easier to separate from the reaction mixture. A very important process on the industrial scale is the hydrogenation of butynediol to butanediol. Butanediol is used for the preparation of tetrahydrofuran (THF), polyTHF, polyesters, etc. The hydrogenation of butynediol to butanediol is generally effected in two stages in industrial scale processes. The second stage here is almost always a fixed bed reactor which is operated under high pressure.

U.S. Pat. No. 6,262,317 (DE 196 41 707 A1) describes the hydrogenation of butyne-1,4-diol with hydrogen in the liquid continuous phase in the presence of a heterogeneous hydrogenation catalyst at temperatures of 20 to 300° C., a pressure of 1 to 200 bar and values of the liquid-side volume-based mass transfer coefficient kLa of 0.1 s⁻¹ to 1 s⁻¹. The reaction can be effected either in the presence of a catalyst suspended in the reaction medium or in a fixed bed reactor operated in cocurrent in cycle gas mode. Inventive example 1 describes a continuous hydrogenation of 100 g/h of a 54% by weight butynediol solution at 35 bar hydrogen and 149° C. over 10 g of a Raney nickel/molybdenum catalyst in suspension in a continuous autoclave, attaining a space-time yield of 0.4 kg of butanediol/(L*h). If the space velocity is increased to a butynediol feed rate of 170 g/h, it is possible to attain a space-time yield of 0.7 kg of butanediol/(L*h), but this also lowered the butanediol yield, and there was a rise in unwanted by-products such as 2-methylbutanediol, n-butanol and n-propanol.

U.S. Pat. No. 3,449,445 describes a process for hydrogenation of butynediol over a Raney nickel suspension catalyst at 50 to 60° C. and 14 to 21 bar in semibatchwise mode. Every Raney nickel catalyst charge can be used for about 20 to 40 batch hydrogenations before it has to be exchanged. On completion of hydrogenation of the butynediol, the catalyst can be settled out. The product is decanted off and filtered and then subjected to further hydrogenation over a fixed catalyst bed at 120 to 140° C. and 138 to 207 bar (2000 to 3000 psig).

In the hydrogenation of butynediol, the content of butenediol, i.e. a partly hydrogenated intermediate, in the product is a measure of the activity of the hydrogenation catalyst and the decrease therein with increasing service life.

DE-A 2 004 611 describes the continuous hydrogenation of butynediol over a Raney nickel fixed bed catalyst at a partial hydrogen pressure of preferably 210 to 360 bar and a temperature of 70 to 145° C. The temperature at the reactor outlet here should not exceed 150° C. in order to avoid excessive formation of by-products (mainly n-butanol). For removal of the heat of reaction, what is described is circulation of the reaction mixture in a circulation stream and withdrawal of heat therefrom. Preferably, the ratio of reaction mixture conducted in the circulation stream to freshly supplied feed is in the range from 10:1 to 40:1, preferably 15:1 to 25:1. As an alternative, other methods of heat removal, such as a stepwise reaction regime with withdrawal of heat between the individual stages, have been described. For the lifetime of the catalyst, a productivity of 325 kg of butanediol/kg of catalyst is reported. The decrease in the catalyst activity over time is manifested in elevated butenediol contents in the product. If the butenediol content that is still tolerable in the product is attained, the original activity of the catalyst can be attained again by increasing the temperature until an outlet temperature of not more than 150° C. has been attained. However, this course of action is limited, as shown by the increasingly shorter time intervals before the next increase in temperature, which indicates rapidly advancing catalyst deactivation. The crude product from the first hydrogenation is subjected to further hydrogenation in each case in a second high-pressure hydrogenation. This allows a reduction in the by-products obtained on average (butenediol, γ-hydroxybutyraldehyde) from 6.6% in the first hydrogenation to 4.1% in the second hydrogenation. It is true that the butenediol content in the crude product is suitable as a measure for the activity or the deactivation of the catalyst. However, what is disadvantageous about this process is that the butenediol content in the crude product has to be measured offline in a complicated manner and the butenediol then still has to be hydrogenated as far as possible to butanediol in a further hydrogenation.

It is known in principle that hydrogenation reactions can be conducted in the presence of carbon monoxide (CO). The CO may firstly be added to the hydrogen used for hydrogenation and/or originate from the feedstocks or the intermediates, by-products or products thereof. If catalysts comprising active components sensitive to CO are used for hydrogenation, a known countermeasure is that of conducting the hydrogenation at a high hydrogen pressure and/or a low catalyst space velocity. Otherwise, the conversion can be incomplete, such that, for example, a postreaction in at least one further reactor is absolutely necessary.

Particularly the adverse effect of CO on the hydrogenation activity of catalysts is known in the literature. DE 26 19 660 uses a palladium catalyst (preferably on a support) for the selective hydrogenation of butyne-1,4-diol to butene-1,4-diol. Before the actual reaction, the palladium catalyst is pretreated here with carbon monoxide (about 200 to 2000 ppm of CO) and about one equivalent of hydrogen and then used for the selective hydrogenation of butynediol to butenediol at a pressure of 1 to 20 bar and a temperature of room temperature to 100° C. It is assumed here that CO binds more strongly to the catalyst surface than butenediol, but less strongly than butynediol. This means that the hydrogenation of butynediol to butenediol is promoted, but the hydrogenation of butenediol to butanediol is inhibited. Only when butynediol has been fully hydrogenated is the butenediol formed hydrogenated further to butanediol. In this case specifically, the inhibiting effect of CO on the catalyst is desirable. In the case of the hydrogenation of butynediol to butanediol, by contrast, it is extremely undesirable.

U.S. Pat. No. 4,361,495 describes a process for regeneration of deactivated supported nickel catalysts that are used in the further hydrogenation of crude butanediol from butynediol hydrogenation. The nickel catalyst used optionally comprises copper and/or manganese and/or molybdenum on a support material such as alumina or silica and has generally been deactivated after the hydrogenation of 500 to 2000 kg of butanediol per kg of catalyst, and so it has to be exchanged. For regeneration, the deactivated catalyst is treated in a hydrogen stream at atmospheric pressure at 200 to 500° C. for about 15 h. For the further hydrogenation of crude butanediol having a carbonyl number of 27 (at 140° C., 138 bar, 6 h), carbonyl numbers of about 0.36 to 0.43 are attained for fresh catalyst, about 2.6 to 3.3 for deactivated catalyst, and 0.52 to 0.59 for a regenerated catalyst. In the context of this application, the carbonyl number attained in the butynediol hydrogenation thus serves as a measure for the activity of the catalyst. A disadvantage of this process is that the carbonyl number likewise has to be measured offline in a complex manner.

DD 265 396 A1 describes a process for preparing butanediol by hydrogenation of butynediol, wherein the reaction is controlled by monitoring the butanol concentration in the hydrogenation product with the aid of the catalyst dosage. In one inventive example, 35% butynediol is hydrogenated at hydrogen pressure 10 bar and 50° C. over a Pd catalyst (catalyst concentration of 60 g/L) to butanediol, wherein the butynediol metering rate was 1 kg of butynediol per kg of Pd catalyst. Over the entire experiment, Pd catalyst was removed continuously from the reaction vessel and fresh catalyst was added. The butanol concentration measured in the hydrogenation output served as a measure for the metering rate: if there was a drop in the butanol content in the hydrogenation product, a greater amount of catalyst was added, whereas less catalyst was added with rising butanol contents. The target corridor for the amount of butanol was 0.03% to 0.3%. Less than 0.1% butenediol was found here in the hydrogenation product. Thus, the butanol concentration served as a closed-loop control parameter in the butynediol hydrogenation in order to intervene in the hydrogenation such that it was possible to keep the product quality constant. Again, complicated offline measurement of the butanol concentration of the hydrogenation output was necessary.

### SUMMARY OF THE INVENTION

It is an object of the present invention to provide an improved process for preparing butane-1,4-diol by catalytic hydrogenation of butyne-1,4-diol, which overcomes as many as possible of the aforementioned disadvantages. More particularly, it should be possible here to implement closed-loop control of at least one of the following parameters in the hydrogenation:
the activity of the catalyst,
the conversion achieved in the hydrogenation,
the selectivity for butane-1,4-diol,
the nature and amount of the by-products obtained,
the product quality, for example the APHA or Hazen color number achieved.

It has been found that this object is achieved when, in the preparation of butane-1,4-diol by catalytic hydrogenation of butyne-1,4-diol, the content of at least one gas selected from CO and CH₄ in the offgas stream is measured and the content of the gas measured in the offgas stream is used for closed-loop control of the hydrogenation.

The invention provides a process for preparing butane-1,4-diol by catalytic hydrogenation of butyne-1,4-diol in a reaction zone with hydrogen in the presence of a heterogeneous hydrogenation catalyst at a temperature in the range from 20 to 300° C. and a pressure in the range from 1 to 300 bar, in which hydrogen is supplied to the reaction zone and an offgas stream is discharged from the reaction zone and the content of at least one gas selected from CO and CH₄ in the offgas stream is measured, wherein
the target value for the content of the gas measured in the offgas stream is fixed, at not more than 5000 ppm by volume for CO and/or at not more than 15% by volume for CH₄,
the actual value for the content of the gas measured in the offgas stream is ascertained,
a control element for influencing a parameter to be controlled in the reaction zone is provided, where the parameter to be controlled for the CO gas measured is selected from the group of increasing the hydrogenation temperature, increasing the energy input, feeding in fresh catalyst, discharging catalyst from the reaction zone, increasing the volume of the offgas stream discharged, increasing the pressure in the reaction zone and reducing the substrate loading per unit catalyst in the reaction zone, and the parameter to be controlled for the CH₄ gas measured is selected from the group of increasing the hydrogenation temperature, discharging catalyst, increasing the volume of the offgas stream discharged and increasing the substrate loading per unit catalyst in the reaction zone,
on attainment of the limit for the deviation in the actual value from the target value, which is not more than 10% of the target value for the measurement of gas, the value for the manipulated variable of the control element (control value) is altered in order to influence the parameter to be controlled in the reaction zone.

### DESCRIPTION OF THE INVENTION

### Closed-Loop Control System

According to the invention, butane-1,4-diol is prepared by catalytic hydrogenation of butyne-1,4-diol in a reaction zone with hydrogen in the presence of a heterogeneous hydrogenation catalyst, in which the content of at least one gas selected from CO and CH₄ in the offgas stream is measured and the content of the gas measured in the offgas stream is used for closed-loop control of the hydrogenation.

By definition, “closed-loop control” refers to an operation in which a parameter, the controlled variable (actual value), is continuously detected, compared with another parameter, the reference variable (target value), and influenced in the manner of assimilation to the reference variable. The closed-loop control deviation as the difference between actual value and target value is sent to the closed-loop controller, which forms a manipulated variable therefrom. The manipulated variable is the output parameter (the position) of the control element used, with the aid of which direct intervention into the control system is effected. The control element may be part of the closed-loop controller, but in many cases is a separate device. The setting or adjustment of the control element controls the process, for example by altering a mass flow or energy flow.

The controlled variable in the process of the invention is the content of a particular gas (CO, CH₄) in the offgas. Examples of control elements are valves, switches, etc. One example of the manipulated variable is the opening state of a valve. The manipulated variable thereof is, for example, the position of the handwheel with which the valve is operated.

If statements are made hereinafter as to the content of a particular gas in the offgas stream, these statements are applicable analogously to the gas space of the reaction zone used for hydrogenation, unless explicitly stated otherwise.

It has been found that compounds such as methane (CH₄), carbon dioxide (CO₂) and carbon monoxide CO are also present in addition to unconverted hydrogen in the offgas stream or in the gas space for the hydrogenation for preparation of butane-1,4-diol from butyne-1,4-diol. It has also been found that, surprisingly, good closed-loop control of the hydrogenation of butyne-1,4-diol is possible when the content of at least one gas selected from CO and CH₄ in the offgas stream is used as controlled variable.

The offgas values can be measured either offline or online, particular preference being given to online measurement.

Measurement of the CO content can be accomplished using standard carbon monoxide sensors that are known to those skilled in the art. These may be based on optochemical detection, infrared measurement, thermal conductivity measurement, exothermicity measurement, electrochemical operations or semiconductor-based sensors. Preference is given to using electrochemical sensors, semiconductor-based sensors or nondispersive infrared sensors.

Measurement of the CH₄ content can likewise be accomplished using standard methane sensors that are known to those skilled in the art. Preference is given to using semiconductor-based sensors or infrared sensors.

A declining catalyst activity or one which is no longer adequate is manifested not only in an elevated CO content or a lower CH₄ content in the offgas stream but also in the incomplete hydrogenation of butynediol and/or rising contents in the product of butene-1,4-diol, 4-hydroxybutyraldehyde, 2-(4-hydroxybutoxy)tetrahydrofuran (called acetal hereinafter) and γ-butyrolactone (called GBL hereinafter). A declining catalyst activity or one which is no longer adequate is likewise manifested in falling pH values and rising APHA numbers in the product stream, which can likewise be measured online and can likewise be used as a measure for the catalyst activity.

### Hydrogenation Catalyst and Reactants

Suitable hydrogenation catalysts for the process of the invention for preparation of butane-1,4-diol by catalytic hydrogenation of butyne-1,4-diol are those catalysts that are suitable for hydrogenation of C-C triple bonds and C-C double bonds to single bonds. They generally contain one or more elements from groups 6 to 11 of the Periodic Table of the Elements. The catalysts preferably comprise at least one element (first metal) selected from Ni, Cu, Fe, Co, Pd, Cr, Mo, Mn, Re, Ru, Pt and Pd. More preferably, the catalysts comprise at least one element (first metal) selected from Ni, Cu, Fe, Co, Pd and Cr. In a specific embodiment, the catalysts comprise Ni.

In a preferred execution, the hydrogenation catalyst additionally comprises at least one promoter element. Preferably, the promoter element is selected from Ti, Ta, Zr, V, Nb, Cr, Mo, W, Mn, Re, Fe, Ru, Co, Rh, Ir, Ni, Pd, Pt, Cu, Ag, Au, Zn, Cd, Ce and Bi. It is possible that the hydrogenation catalyst comprises at least one promoter element which simultaneously fulfills the definition of a first metal in the context of the invention. Promoter elements of this kind are selected from Ni, Fe, Co, Cu, Cr, Pt, Ag, Au, Pd, Mn, Re, Ru, Rh and Ir. In this case, the hydrogenation catalyst, based on the reduced metallic form, contains a majority (i.e. more than 50% by weight) of the first metal and a minority (i.e. less than 50% by weight) of a different metal as promoter element. In stating the total amount of the first metal that the hydrogenation catalyst comprises, however, all metals that fulfill the definition of a first metal in the context of the invention are calculated with their full proportion by weight (irrespective of whether they act as hydrogenation-active component or as promoter). Preferably, the hydrogenation catalyst comprises exclusively a promoter element or more than one promoter element selected from Ti, Ta, Zr, V, Mo, W, Bi and Ce. Preferably, the hydrogenation catalyst comprises Mo as promoter element. In a specific embodiment, the hydrogenation catalyst comprises Mo as the sole promoter element.

Preferably, the hydrogenation catalyst, based on the reduced metallic form, comprises a first metal in an amount of 0.1% to 100% by weight, preferably 0.2% to 99.5% by weight, more preferably 0.5% to 99% by weight.

The promoter content of the catalyst is generally up to 25% by weight, preferably 0.001% to 15% by weight, more preferably 0.01% to 13% by weight.

Suitable heterogeneous hydrogenation catalysts are precipitated catalysts, supported catalysts or Raney metal catalysts. Typically, Raney catalysts are alloys comprising at least one catalytically active metal and at least one alloy component soluble (leachable) in alkalis. Typical catalytically active metals are, for example, Ni, Fe, Co, Cu, Cr, Pt, Ag, Au and Pd, and typical leachable alloy components are, for example, Al, Zn and Si. Raney metal catalysts of this kind and processes for preparation thereof are described, for example, in U.S. Pat. Nos. 1,628,190, 1,915,473, 1,563,587. Before they are used in heterogeneously catalyzed chemical reactions, specifically in a hydrogenation reaction, Raney metal alloys generally have to be subjected to an activation. Standard processes for activating Raney metal catalysts comprise the grinding of the alloy to give a fine powder if it is not already in powder form as produced. For activation, the powder is subjected to a treatment with an aqueous alkali, with partial removal of the leachable metal from the alloy, leaving the highly active non-leachable metal.

Support materials used for supported catalysts may be aluminum oxides, titanium dioxides, zirconium dioxide, silicon dioxide, aluminas, e.g. montmorillonites, silicates such as magnesium or aluminum silicates, zeolites and activated carbons. Preferred support materials are aluminum oxides, titanium dioxides, silicon dioxide, zirconium dioxide and activated carbons. It is of course also possible to use mixtures of different support materials as support for catalysts employable in the process of the invention. These catalysts can be used either in the form of shaped catalyst bodies, for example in the form of spheres, cylinders, rings or spirals, or in the form of powders. Preference is given to using the catalysts in the form of shaped bodies. Suitable catalysts for the hydrogenation are known, for example, from DE-A 12 85 992, DE-A 25 36 273, EP-A 177 912, EP-A 394 841, EP-A 394 842, U.S. Pat. No. 5,068,468, DE-A 1 641 707 and EP-A 922 689. U.S. Pat. No. 6,262,317 (DE 196 41 707 A1) describes the production of fixed bed reactors by directly coating structured packings as typically used in bubble columns with catalytically active substances.

In a specific execution, “monolithic” shaped bodies are used as catalyst supports. Monolithic shaped bodies are structured shaped bodies suitable for production of immobile structured fixed beds. By contrast with particulate catalysts and catalyst supports, it is possible to use monolithic shaped bodies to create essentially coherent and seamless fixed beds. The monolithic shaped bodies used in the process of the invention are preferably in the form of a foam, mesh, woven fabric, loop-drawn knitted fabric, loop-formed knitted fabric or another monolith. The term “monolithic shaped body” in the context of the invention also includes structures known as “honeycomb catalysts”. In a specific embodiment, the shaped bodies are in the form of a foam. Suitable monolithic shaped bodies are as described, for example, in EP-A-0 068 862, EP-A-0 198 435, EP-A 201 614 and EP-A 448 884. EP 2 764 916 A1 describes hydrogenation catalysts based on shaped catalyst bodies in the form of foams.

The hydrogenation catalysts can be used in a fixed bed or in suspension. When the catalysts are arranged in the form of a fixed bed, the reactor can be operated in trickle mode or in liquid phase mode. In a specific execution, the catalyst is arranged in the form of a fixed bed and is operated in an upward cocurrent flow of liquid and gas. It is especially then the liquid and not the gas that is present as the continuous phase.

The process of the invention is preferably conducted with technical grade butyne-1,4-diol. This is in the form of an aqueous solution and may comprise insoluble or dissolved constituents from the butyne-1,4-diol synthesis. These include, for example, copper compounds, bismuth compounds, aluminum compounds or silicon compounds. It is of course also possible to use purified butyne-1,4-diol in the process of the invention. Crude butyne-1,4-diol is purified, for example, by distillation. Butyne-1,4-diol can be prepared on the industrial scale from acetylene and aqueous formaldehyde and is typically hydrogenated as an aqueous 30% to 60% by weight solution. Alternatively, it can be hydrogenated in other solvents, for example alcohols such as methanol, ethanol, propanol, butanol or butane-1,4-diol. The hydrogen required for the hydrogenation is preferably used in pure form, but it may also comprise additions of other gases, for example methane and carbon monoxide.

### Hydrogenation Conditions

For the hydrogenation by the process of the invention, suitable reactors in principle are pressure-resistant reactors as customarily used for exothermic heterogeneous reactions involving feeding in one gaseous and one liquid reactant. These include the generally customary reactors for gas-liquid reactions, for example tubular reactors, shell and tube reactors and gas circulation reactors. A specific embodiment of the tubular reactors is that of shaft reactors. Reactors of this kind are known in principle to the person skilled in the art. More particularly, a cylindrical reactor having a vertical longitudinal axis is used, having, at the base or top of the reactor, an inlet apparatus or a plurality of inlet apparatuses for feeding in a reactant mixture comprising at least one gaseous and at least one liquid component. Substreams of the gaseous and/or the liquid reactant can be fed to the reactor additionally, if desired, via at least one further feed apparatus. The reaction mixture of the hydrogenation in the reactor generally takes the form of a biphasic mixture having a liquid phase and a gaseous phase.

The processes of the invention are specifically suitable for hydrogenations which are to be conducted on an industrial scale. Preferably, the reactor in that case has an internal volume in the range from 0.1 to 100 m³, preferably from 0.5 to 80 m³. The term “internal volume” here relates to the volume including the fixed catalyst bed(s) present in the reactor and any further internals present. The technical advantages associated with the process of the invention are of course also manifested even in reactors with a smaller internal volume.

A biphasic gas/liquid mixture generally flows through the reaction zone. The reactants are generally fed into the reaction zone in the form of a liquid feed comprising butyne-1,4-diol and water, and a gaseous hydrogen feed. The reactants can be fed into the reactor separately or in premixed form in a customary manner. It is possible, for example, to use mixing nozzles into which the liquid feed and the gas feed are fed. It is possible to operate the process of the invention with a liquid circulation stream and/or a gaseous circulation stream. In that case, the recycling of the liquid circulation stream into the reaction zone can be effected together with the liquid feed, and the recycling of the gaseous circulation stream together with the fresh hydrogen feed. In this case too, separate feeding of individual streams and mixing of gaseous and liquid components is possible.

A biphasic gas/liquid mixture exits from the reaction zone. It is possible to discharge the gas leaving the reaction zone and the liquid leaving the reaction zone in the form of separate streams (offgas and liquid output). It is additionally possible to discharge gas and liquid together and only then to undertake a gas/liquid separation.

For avoidance of accumulation of inert constituents, it is possible to remove a substream from the offgas and discharge it. In a specific embodiment, the offgas is at least partly conducted in a circulation stream (cycle gas mode). In cycle gas mode, the offgas leaving the reaction zone, optionally after discharge of a substream for avoidance of the accumulation of inert constituents and optionally after supplementation with fresh hydrogen, is recycled into the reactor. The recycling is effected, for example, via a compressor. It is possible to conduct the entire cycle gas volume or a portion thereof through a motive jet compressor. In this preferred embodiment, the cycle gas compressor is replaced by an inexpensive nozzle.

The liquid output is at least partly subjected to the isolation of a product stream comprising the crude butane-1,4-diol. In a specific embodiment, the liquid output is at least partly conducted in a circulation stream. This involves recycling the liquid output into the reactor after discharge of a substream as product stream and optionally after passage through a heat exchanger to remove heat of reaction.

According to the invention, the content of at least one gas selected from CO and CH₄ in the offgas stream is measured. If there is already a separation of the biphasic gas/liquid mixture exiting from the reaction zone in the reactor, the gas content can be measured in the gas phase present in the reactor before it is discharged as offgas stream. It is also possible that the gas content is measured in the offgas stream from the reactor. In cycle gas mode, it is also possible that the gas content is measured in the cycle gas before fresh hydrogen is fed in. When gas and liquid are discharged together from the reactor and a gas/liquid separation is only then undertaken, the gas content can be measured in the gas phase obtained after the phase separation of the gas/liquid output.

The temperature in the hydrogenation is preferably within a range from 20 to 300° C., more preferably from 40 to 250° C.

The absolute pressure in the hydrogenation is preferably within a range from 1 to 350 bar, more preferably within a range from 5 to 300 bar.

If the hydrogenation catalyst is used in the form of a fixed bed, the temperature in the hydrogenation is preferably within a range from 30 to 300° C., more preferably from 50 to 250° C., especially from 70 to 220° C. If the hydrogenation catalyst is used in the form of a fixed bed, the pressure in the hydrogenation is preferably within a range from 25 to 350 bar, more preferably from 100 to 300 bar, especially from 150 to 300 bar.

If the hydrogenation catalyst is used in the form of a suspension, the temperature in the hydrogenation is preferably within a range from 20 to 300° C., more preferably from 60 to 200° C., especially from 120° C. to 180° C.

If the hydrogenation catalyst is used in the form of a suspension, the pressure in the hydrogenation is preferably within a range from 1 to 200 bar, more preferably from 5 to 150 bar, especially from 20 to 100 bar.

The molar ratio of hydrogen fed to the reaction zone to butyne-1,4-diol fed to the reaction zone is preferably at least 2:1.

The molar ratio of hydrogen fed to the reaction zone to butyne-1,4-diol fed to the reaction zone is preferably within a range of 2.01:1 to 4:1, more preferably 2.01:1 to 3:1 and most preferably 2.01:1 to 2.6:1. Specifically, the molar ratio of hydrogen fed to the reaction zone to butyne-1,4-diol fed to the reaction zone is 2.2:1 to 2.4:1.

In a preferred embodiment, the reaction mixture of the hydrogenation is at least partly conducted in a liquid circulation stream. In that case, the molar ratio of fresh hydrogen fed to the reaction zone to fresh butyne-1,4-diol fed to the reaction zone is preferably at least 2:1.

If the reaction mixture for the hydrogenation is conducted at least partly in a liquid circulation stream, the molar ratio of fresh hydrogen fed to the reaction zone to fresh butyne-1,4-diol fed to the reaction zone is preferably within a range of 2.01:1 to 4:1, more preferably 2.01:1 to 3:1 and most preferably 2.01:1 to 2.6:1. Specifically, the molar ratio of fresh hydrogen fed to the reaction zone to fresh butyne-1,4-diol fed to the reaction zone is 2.2:1 to 2.4:1.

If the reaction mixture for the hydrogenation is conducted at least partly in a liquid circulation stream, the ratio of gas stream fed to the reactor to gas stream leaving the reactor is preferably within a range from 0.99:1 to 0.4:1. In other words, at least 60% of the gas supplied leaves the reactor system. Thus, in cycle gas mode, it is possible to avoid accumulation of unwanted components such as CO in the gas stream.

Preferably, the conversion of butyne-1,4-diol is 90% to 100%, more preferably 98% to 100%, especially 99.5% to 100%.

In general, the yield of butane-1,4-diol achieved by catalytic hydrogenation of butyne-1,4-diol is lower than the conversion of butyne-1,4-diol, since there is also formation of further by-products, for example propanol, butanol, hydroxybutyraldehyde, acetal, γ-butyrolactone (GBL). At the same time, the process of the invention enables high selectivity for the butane-1,4-diol target compound. More particularly, it is possible to avoid undesirably high formation of butenediol and of hydroxybutyraldehyde. Elevated butenediol contents are generally associated with elevated contents of hydroxybutyraldehyde, and the latter in turn with an elevated content of methylbutanediol and acetal. Thus, elevated butenediol contents lead not only to poor product quality, but also suggest declining catalyst activity. Preferably, the liquid reaction mixture present in the reaction zone has a butenediol content of not more than 7000 ppm by weight.

### Closed-Loop Control Via the CO Content in the Offgas

In a first embodiment (variant 1), in the process of the invention, the content of CO in the offgas stream is measured and it is ensured by means of the measures described in detail hereinafter that the CO content does not exceed the limits specified. Thus, closed-loop control of the preparation of butane-1,4-diol by catalytic hydrogenation of butyne-1,4-diol at least is possible in relation to at least one of the following properties:
the activity of the catalyst,
the conversion achieved in the hydrogenation,
the selectivity for butane-1,4-diol,
the nature and amount of the by-products obtained,
the product quality, for example the APHA or Hazen color number achieved.

Preferably, in this variant, the hydrogenation is effected at a temperature in the range from 100 to 300° C., more preferably from 100 to 200° C., especially from 110 to 180° C.

Preferably, the target value for the CO content in the offgas is not more than 5000 ppm by volume, more preferably not more than 2000 ppm by volume, particularly not more than 1000 ppm by volume and especially not more than 800 ppm by volume.

Preferably, the target value for the CO content is within a range from 0.05 to 5000 ppm by volume, more preferably within a range from 0.1 to 2000 ppm by volume, particularly within a range from 0.1 to 1000 ppm by volume and especially within a range from 0.1 to 800 ppm by volume.

Preferably, the limit for the deviation in the actual value for the CO content in the offgas from the target value is not more than 10%, more preferably not more than 5%, based on the target value.

Typical CO contents in the offgas from the catalytic hydrogenation of butyne-1,4-diol for preparation of butane-1,4-diol at the start in the case of hydrogenation with fresh catalyst are within a range from, for example, 0.01 to 50 ppm. With increasing service life of the catalyst, there is a decrease in the activity of the catalyst and generally a gradual rise in the contents of CO in the offgas. Typical values for the rise in the CO content in the offgas stream, depending on catalyst activity, catalyst age, space velocity and temperature, are about 1 to 50 ppm per day. In principle, it is difficult to keep the selectivity, the conversion and/or the product quality in the hydrogenation at an acceptable level with high CO contents in the offgas as well. One possible measure would be the reduction of the butynediol loading per unit catalyst (expressed in kg(butynediol)/(kg of catalyst)*h), in which case there is also a drop in the CO content in the offgas. A conceivable example would be the reduction of the butynediol loading per catalyst unit by 1% to 80%, especially by 5% to 50%, very particularly by 5% to 30%. However, a disadvantage of such an approach is that a reduction in the catalyst space velocity is undesirable for economic reasons, since this results in a reduced space-time yield. Moreover, this means that only the residual catalyst activity still present is utilized.

Preference is therefore given to a process in which the content of CO in the offgas stream is measured and, on attainment of the limit for the deviation of the actual value of the CO content of the offgas stream from the target value, at least one of the following parameters in the reaction zone is controlled:
increasing the hydrogenation temperature,
increasing the energy input,
feeding in fresh catalyst,
discharging catalyst from the reaction zone,
increasing the volume of the offgas stream discharged,
increasing the pressure in the reaction zone,
reducing the substrate loading per unit catalyst in the reaction zone.

The above-described measures can each be conducted individually or in any combinations. In a specific execution, discharge of catalyst from the reaction zone is not conducted as the sole measure. In that case, preference is given to feeding fresh catalyst into the reaction zone. It is thus possible to avoid an increase in the substrate loading per unit catalyst in the reaction zone.

In principle, closed-loop control interventions with any frequency are also possible until the CO content can no longer be kept within an acceptable range and, for example, the entire catalyst has to be exchanged.

With the measurement devices available industrially for determination of the CO content in the offgas stream, the hydrogenation performance can be determined within very short time intervals, i.e. within the range of minutes or even seconds. In any case, it can be ensured that the interval between two measurements is much shorter than the response time of the reaction system to a closed-loop control intervention. In the context of the invention, an “online measurement” refers to a measurement which is effected without extractive sampling and wherein the data are measured directly at their site of origin.

With an online measurement of the offgas values, the hydrogenation performance of the system can to some degree be followed in real time. What is advantageous about an online measurement compared to an offline measurement is that the measures listed above can be taken without loss of time. This is especially advantageous in the case of performance of the hydrogenations with suspended catalyst. If the hydrogenation does not run in an ideal manner or the hydrogenation is disrupted in situ, for example by agglomerated catalyst, this can be seen rapidly from the CO offgas values. In such a case, rates of 1 to 1000 ppm per hour are observed for the rise in CO. In the case of online measurement of the CO content in the offgas stream, it is then possible to intervene immediately. This has not just economic advantages but in particular also safety-related advantages. In the case of a rapid rise in the CO contents, the hydrogenation no longer proceeds to completion, and so intervention into the system is advisable (for example by reduction in the space velocity or shutdown).

Preferably, the volume ratio of CO:CO₂ is not more than 1:500, especially 1:400 and most preferably 1:300.

Preferably, the limit for the deviation in the actual value for the CO content in the offgas from the target value is not more than 10%, more preferably not more than 5%, based on the target value.

Preference is given to a process in which the content of CO in the offgas stream is measured and, on attainment of the limit for the deviation of the actual value of the CO content of the offgas stream from the target value, at least one of the following parameters in the reaction zone is controlled.

An increase in the hydrogenation temperature is preferably by 1 to 10° C., more preferably by 1 to 8° C., especially by 1 to 5° C.

When the energy introduced into the reaction zone is increased, it is increased preferably by 2% to 30%, more preferably by 2% to 20%, especially by 2% to 10%. The energy input into the reaction zone can be increased, for example, by increasing the stirring energy, the energy introduced in the circulation stream by pump circulation, the energy introduced by gas injection, etc.

When fresh catalyst is fed into the reaction zone, preferably 1% to 50% by weight, more preferably 1% to 30% by weight, especially 1% to 10% by weight, of fresh catalyst is fed in, based on the total weight of the catalyst previously present in the reaction zone.

When catalyst is discharged from the reaction zone, preferably 1% to 50% by weight, more preferably 1% to 30% by weight, especially 1% to 10% by weight, of the catalyst present in the reaction zone is discharged, based on the total weight of the catalyst present in the reaction zone.

When the volume of the offgas stream discharged from the reaction zone is increased, it is preferably increased by 10 to 500 mol %, more preferably by 10 to 200 mol %, especially by 10 to 100 mol %.

When the pressure in the reaction zone is increased, it is preferably increased by 1 to 30 bar, more preferably by 1 to 20 bar, especially by 1 to 10 bar.

When the substrate loading per unit catalyst (in kg(substrate)/(kg of catalyst)×h) is reduced, it is preferably reduced by 1% to 80%, more preferably by 3% to 50%, especially by 5% to 30%.

### Closed-Loop Control Via the CH₄ Content in the Offgas

In a second embodiment (variant 2), in the process of the invention, the content of CH₄ in the offgas stream is measured and it is ensured by means of the measures described in detail hereinafter that the CH₄ content does not exceed the limits specified. Thus, closed-loop control of the preparation of butane-1,4-diol by catalytic hydrogenation of butyne-1,4-diol at least is possible in relation to at least one of the following properties:
the activity of the catalyst,
the conversion achieved in the hydrogenation,
the selectivity for butane-1,4-diol,
the nature and amount of the by-products obtained,
the product quality, for example the APHA or Hazen color number achieved.

As well as CO, the content of CH₄ in the offgas stream can also be determined efficiently by means of one of the above-described measurement devices. Preferably, the CH₄ content in the offgas stream is measured by an online IR measurement. By contrast with CO, methane is not a catalyst poison, but is a gas which is inert under the reaction conditions of the hydrogenation of the invention.

The target value for the CH₄ content in the offgas is preferably not more than 15% by volume. Preferably, the target value for the CH₄ content in the offgas is within a range from 1% to 15% by volume. These values are generally applicable irrespective of the offgas volumes and hydrogen excesses used in the process.

The CH₄ content in the offgas stream which is suitable in the context of the process of the invention depends on what offgas volumes are used and in what excess the hydrogen is used compared to the amount theoretically required for hydrogenation of the butyne-1,4-diol. Thus, it is also possible in principle that the CH₄ content in the offgas is more than 15% by volume if this is compensated for by a simultaneous increase in the partial hydrogen pressure.

Preferably, the limit for the deviation in the actual value for the CH₄ content in the offgas from the target value is not more than 10%, more preferably not more than 5%, based on the target value.

Preference is given to a process in which the content of CH₄ in the offgas stream is measured and, on attainment of the limit for the deviation of the actual value of the CH₄ content of the offgas stream from the target value, at least one of the following parameters in the reaction zone is controlled:
reducing the hydrogenation temperature,
discharging catalyst,
increasing the volume of the offgas stream discharged,
increasing the substrate loading per unit catalyst in the reaction zone.

A reduction in the hydrogenation temperature is preferably by 1 to 10° C., more preferably by 1 to 8° C., especially by 1 to 5° C.

When catalyst is discharged from the reaction zone, preferably 1% to 50% by weight, more preferably 1% to 30% by weight, especially 1% to 10% by weight, of the catalyst present in the reaction zone is discharged, based on the total weight of the catalyst present in the reaction zone.

When the volume of the offgas stream discharged from the reaction zone is increased, it is preferably increased by 10 to 500 mol %, more preferably by 10 to 200 mol %, especially by 10 to 100 mol %.

When the substrate loading per unit catalyst (in kg(substrate)/(kg of catalyst)×h) is increased, it is preferably reduced by 1% to 80%, more preferably by 3% to 50%, especially by 5% to 30%.

With increasing service life of the catalyst, there is a reduction in the activity thereof, which also decreases the amount of methane in the offgas. With decreasing activity of the catalyst, by contrast, however, there is a rise in the amount of CO in the offgas, which in turn adversely affects the product quality. The measures presented in the context of the present invention can control the hydrogenation and keep at least one, preferably more than one, especially all, of the aforementioned process parameters within the desired range. If the methane value is too high, the measures described here can be taken in order to lower the activity of the catalyst or to adjust the space velocity, which means that less product of value is destroyed. If, by contrast, the CO content is too high, the measures described here can be taken in order to increase or to adjust the activity of the catalyst, in order to maintain the product quality. The product quality of the crude butane-1,4-diol obtained by the process of the invention is sufficiently high that no further hydrogenation is necessary for many applications.

The examples which follow serve to illustrate the invention, but without restricting it in any way.

### EXAMPLES

The measurement method used is an IR measurement. The spectrometer is an IR spectrometer of the Thermo Fisher Protege 460 type. The measurement cell is a 2 m multipass cell from Thermo Fisher. The measurement was effected at room temperature. The evaluation for CO was effected at 2175 cm⁻¹, that for CO₂ at 2380 cm⁻¹, and that for CH₄ at 3150 cm⁻¹.

### Example 1: (Measurement of the CO Content and Control by Reduction of the Substrate Loading Per Unit Catalyst)

A 2 L autoclave filled to 1 L was charged with 100 g of Raney nickel-molybdenum catalyst and heated up to 160° C. while stirring, and H₂ was injected to 45 bar. An aqueous, approximately 50% by weight butynediol solution was run into the autoclave at a feed rate of 800 to 1000 g (butynediol solution)/h, and a correspondingly high product flow rate was discharged from the reactor. The H₂ feed rate corresponded to about 2.2 mol of H₂ per mole of butynediol. After operation for about 400 hours, at a feed rate of 800 g (butynediol solution)/h, about 60 ppm of CO, 1600 ppm of CO₂ and 14% by volume of CH₄ were found in the offgas. A GC analysis of the liquid gave 1.54% methanol, 1.26% propanol, 0.94% butanol, 95% butane-1,4-diol (BDO), 1000 ppm 2-methylbutane-1,4-diol (MBDO), 310 ppm acetal and 130 ppm butenediol (BED) at a pH of 7.2 and an APHA number (determined according to ASTM D1209) of 120. Once the feed rate had been reduced from 800 g (butynediol solution)/h to 500 g (butynediol solution)/h and the H₂ feed rate had been increased to 2.4 mol of H₂ per mole of butynediol, offgas values of 24 ppm of CO, 297 ppm of CO₂ and 12.3% by volume of methane were obtained. A GC analysis of the liquid gave 1.68% methanol, 1.70% propanol, 1.12% butanol, 94.1% BDO, 800 ppm MBDO, 100 ppm acetal and no butenediol at a pH of 7.4 and an APHA number of 105. After the space velocity had been increased again to 800 to 1000 (butynediol solution)/h and a total run time of 700 h, 190 ppm CO, 5200 ppm CO₂ and 10.7% by volume CH₄ were found in the offgas, with a composition of the liquid of 1.92% methanol, 1.36% propanol, 1.76% butanol, 93.4% BDO, 1300 ppm MBDO, 1100 ppm acetal and 420 ppm BED at a pH of 6.8 and an APHA number of 168.

### Example 2 (Hydrogenation of Butynediol, Measurement of CH₄ Content and Control by Reduction of the Hydrogenation Temperature)

The reaction conditions correspond to those in example 1. The butynediol feed rate was 900 g (butynediol solution)/h. On the first day, at a temperature of 160° C., the amount of methane in the offgas was 30% by volume, while the CO content in the offgas was 0.1 ppm. The propanol content in the product was 2%. After a reduction in the temperature by 10° C., it was possible to reduce the methane content in the offgas to 15% by volume. At the same time, the propanol content in the product fell to 1.5%, and so the butanediol content rose from 95% to 95.5%. The rest consisted essentially of methanol (from formaldehyde), butanol, GBL and further by-products.

### Example 3 (Hydrogenation of Butynediol, Measurement of CO Content and Control by Temperature Increase)

The reaction conditions correspond to those in example 2. The butynediol feed rate was 900 g (butynediol solution)/h at a temperature of 150° C. After a run time of 300 h, there was a rise in the CO content in the offgas from 0.1 ppm to 170 ppm in the offgas, while the CH₄ content fell from 15% by volume to 11% by volume. The content of butenediol rose from <5 ppm to 140 ppm and the acetal content rose from 300 ppm to 600 ppm in the output. After the temperature had been increased from 150° C. to 152° C., there was a drop in the content of CO in the offgas from 170 ppm to 30 ppm, while the methane content rose from 11% by volume to 12% by volume. The butenediol content fell from 140 ppm to 10 ppm and the acetal content fell from 600 ppm in the output to 250 ppm. As soon as the limit of 170 ppm of CO in the offgas had been exceeded, the temperature was increased by 2° C.

### Example 4 (Measurement of CO Content and Control by Catalyst Discharge)

The reaction conditions correspond to those in example 3. The butynediol feed rate was 900 g (butynediol solution)/h. After multiple increases in temperature, at a temperature of 160° C., the limit of 170 ppm of CO in the offgas was again exceeded. Subsequently, via a lock, 10 g of the spent catalyst were discharged and 10 g of fresh catalyst were added to the system. Subsequently, owing to the elevated catalyst activity available, there was a drop in the CO content in the offgas from 170 ppm to 27 ppm and a drop in the butenediol content in the output from 120 ppm to 19 ppm, while there was a drop in the acetal content from 780 ppm to 326 ppm. After the catalyst injection, the methane content increased from 7% by volume to 8.2% by volume.

1. A process for preparing butane-1,4-diol by catalytic hydrogenation of butyne-1,4-diol in a reaction zone with hydrogen in the presence of a heterogeneous hydrogenation catalyst at a temperature in the range from 20 to 300° C. and a pressure in the range from 1 to 350 bar, in which hydrogen is supplied to the reaction zone and an offgas stream is discharged from the reaction zone and the content of at least one gas selected from CO and CH₄ in the offgas stream is measured, wherein:
a target value for the content of the gas measured in the offgas stream is fixed, at not more than 5000 ppm by volume for CO and/or at not more than 15% by volume for CH₄,
an actual value for the content of the gas measured in the offgas stream is ascertained,
a control element for influencing a parameter to be controlled in the reaction zone is provided, where the parameter to be controlled for the CO gas measured is selected from the group of increasing the hydrogenation temperature, increasing the energy input, feeding in fresh catalyst, discharging catalyst from the reaction zone, increasing the volume of the offgas stream discharged, increasing the pressure in the reaction zone and reducing the substrate loading per unit catalyst in the reaction zone, and the parameter to be controlled for the CH₄ gas measured is selected from the group of increasing the hydrogenation temperature, discharging catalyst, increasing the volume of the offgas stream discharged and increasing the substrate loading per unit catalyst in the reaction zone,
on attainment of a limit for the deviation in the actual value from the target value, which is not more than 10% of the taget value for the measurement of gas, control value for the manipulated variable of the control element is altered in order to influence the parameter to be controlled in the reaction zone.

2. The process according to claim 1, wherein the hydrogenation is effected at a temperature in the range from 100 to 300° C., and the content of CO in the offgas stream is measured.

3. The process according to claim 1, wherein the target value for the CO content in the offgas is not more than 2000 ppm by volume.

4. The process according to claim 2, wherein the limit for the deviation in the actual value for the CO content in the offgas from the target value is not more than 5%, based on the target value.

5. (canceled)

6. The process according to claim 1, wherein the hydrogenation temperature is increased by 1 to 10° C., when the limit for the deviation in the actual value of the CO content has been attained, or else is lowered when the limit for the deviation in the actual value of the CH₄ content has been attained.

7. The process according to claim 1, wherein the energy introduced into the reaction zone is increased by 2% to 30%, when the limit for the deviation in the actual value of the CO content has been attained.

8. (canceled)

9. The process according to claim 1, wherein 1% to 50% by weight of the catalyst present in the reaction zone, based on the total weight of the catalyst present in the reaction zone, is discharged therefrom.

10. The process according to claim 1, wherein the volume of the offgas stream discharged from the reaction zone is increased by 10 to 500 mol %.

11. The process according to claim 1, wherein the pressure in the reaction zone, when the limit for the deviation in the actual value of the CO content has been attained, is increased by 1 to 30 bar.

12. The process according to claim 1, wherein the substrate loading per unit catalyst (in kg(substrate)/(kg of catalyst)×h), when the limit for the deviation in the actual value of the CO content has been attained, is reduced by 1% to 80% or is increased when the limit for the deviation in the actual value of the CH₄ content has been attained.

13. (canceled)

14. The process according to claim 1, wherein the limit for the deviation in the actual value for the CH₄ content in the offgas from the target value is not more than 5%, based on the target value.

15. (canceled)

16. The process according to claim 2, wherein the hydrogenation is effected at a temperature in the range from 100 to 200° C.

17. The process according to claim 3, wherein the target value for the CO content in the offgas is not more than 1000 ppm by volume.

18. The process according to claim 6, wherein the hydrogenation temperature is increased by 1 to 8° C. when the limit for the deviation in the actual value of the CO content has been attained.

19. The process according to claim 7, wherein the energy introduced into the reaction zone is increased by 2% to 20% when the limit for the deviation in the actual value of the CO content has been attained.

20. The process according to claim 9, wherein 1% to 30% by weight of the catalyst present in the reaction zone, based on the total weight of the catalyst present in the reaction zone, is discharged therefrom.

21. The process according to claim 10, wherein the volume of the offgas stream discharged from the reaction zone is increased by 10 to 200 mol %.

22. The process according to claim 11, wherein the pressure in the reaction zone, when the limit for the deviation in the actual value of the CO content has been attained, is increased by 1 to 20 bar.

23. The process according to claim 12, wherein the substrate loading per unit catalyst (in kg(substrate)/(kg of catalyst)×h), when the limit for the deviation in the actual value of the CO content has been attained, is reduced by 3% to 50%.
